(19) United States
(12) Patent Application Publication
(10) Pub. No.: US 2015/0268219 A1
(43) Pub. Date: Sep. 24, 2015

(54) Method for Renal Transplantation

(71) Applicants: Universite Paris Diderot Paris 7, Paris (FR); Centre National de la Recherche Scientifique (CNRS), Paris (FR); Institut National de la Sante et de la Recherche Medicale (INSERM), Paris (FR); Universite Paris Descartes, Paris (FR); Assistance Publique - Hopitaux de Paris, Paris (FR)

(72) Inventors: Ivan CRUZ MOURA, Montrouge (FR); Celine VAUGIER, Paris (FR); Martin FLAMANT, Colombes (FR); Olivier HERMINE, Palaiseau (FR)

(21) Appl. No.: 14/223,093

(22) Filed: Mar. 24, 2014

(51) Int. Cl. G01N 33/483; A61K 35/22; A61K 33/26

(52) U.S. Cl. CPC G01N 33/4833; A61K 33/26; A61K 35/22

(57) ABSTRACT

The present invention is in the technical field of transplantation, and more particularly relates to the prognosis of a renal transplantation, and to the prevention of renal ischemia and reperfusion injury (IRI) prior to renal transplantation. The invention is more particularly based on the findings that the circulating level of ferritin in a patient is predictive of the outcome of renal transplantation, and that an iron administration to said patient prior to transplantation can prevent IRI.

### INTRODUCTION

The present invention is in the technical field of transplantation, and more particularly relates to the prognosis of a renal transplantation, and to the prevention of renal ischemia and reperfusion injury (IRI) prior to renal transplantation. The invention is more particularly based on the findings that the circulating level of ferritin in a patient is predictive of the outcome of renal transplantation, and that an iron administration to said patient prior to transplantation can prevent IRI, a common complication of renal transplantation which contributes to delayed graft function (DGF) and can be notably observed in end stage renal disease (ESRD) patients.

DGF is an early complication of renal transplantation impacting both short- and long-term graft survival (Hariharan et al., 2000; Tilney et al., 1997; Herrero-Fresneda et al., 2003). This condition is in most cases a consequence of acute tubular necrosis (ATN) frequently caused by ischemia-reperfusion injury (IRI). Known risk factors interfering in DGF includes cold ischemia time, cold storage preservation, age and sex gender of the donor, the use of kidney allografts from circulatory death donors and recipient's age. However, these parameters are not fully capable of predicting the outcome of kidney transplants. Therefore, the investigation of new factors predicting allograft function could contribute to extend kidney transplants survival and/or function.

In addition, despite improvements in prevention strategies such as the reduction of cold ischemia time, recipient fluid content management, the use of immunosuppressive drugs, and/or the amelioration of the composition of the organ preservation solution (Perico et al., 2004), DGF still occurs in allografted recipients. A better understanding of factors capable of modulating the physiopathology of IRI is therefore critical in order to develop new specific therapies to extend graft survival and/or function.

Thus, there is an urgent need in the art to provide a new reliable, and preferably non-invasive, method capable of predicting the functionality and/or survival of a kidney transplant, as well as a novel therapeutic strategy to improve the outcome of kidney transplants.

The above discussed needs are addressed by the present invention, which reports herein the results from investigations conducted on a cohort of kidney allograft recipients (147), as well as on an experimental animal model of post-ischemic renal injury.

The inventors have surprisingly shown that an elevated serum ferritin level was correlated with an improved renal function outcome in patients receiving a kidney allograft, and demonstrated that a constitutive systemic iron overload offered a protection against IRI and reduced inflammatory responses occurring in post-ischemic injury. In particular, the inventors showed that macrophages differentiated in vitro in the presence of supra-physiological iron concentrations presented reduced expression of genes involved in NF-κB and inflammatory pathways activation, and that those cells were unable to reconstitute IRI in macrophage-depleted animals.

These observations are in stark contrast with the reports currently available in the literature. Indeed, a large literature supports the pro-inflammatory role of an excess of circulating iron through its participation in Fenton reaction, the production of reactive oxygen species (ROS) and the activation of NF-κB and mitogen-activated protein kinase (MAPK) pathways (Bhattacharyya et al., 2012; Deb et al., 2009; Munoz et al., 2006; Yuan et al., 2004; Pham et al., 2004). In a recent report, iron overload was associated with the emergence of an uncontrolled proinflammatory M1 macrophage population. These cells presented enhanced TNF-alpha and ROS release which were found to be pathological in chronic venous leg ulcers (Sindrilaru et al., 2011).

To the best of Applicant's knowledge, this is the first study demonstrating that an increased baseline serum ferritin level is associated with a favorable graft outcome, and that iron is a critical modulator of post-ischemic injury in both mice and men, which implies that iron overload can be used to improve renal transplantation outcome.

Based on the findings disclosed herein, the present invention provides for the first time a reliable, accurate and quick and easy method to perform for prognosing a renal transplantation, which is based on the determination of the circulating level of ferritin in a patient prior to transplantation. The invention further provides a prophylactic/therapeutic method of kidney ischemia-reperfusion injury, as well as a renal transplantation method, which comprises the administration to a patient in need thereof of a biological material capable of increasing the circulating ferritin level so as to reach a critical level.

### DETAILED DESCRIPTION OF THE INVENTION

Unless stated otherwise, scientific and technical terms used in connection with the present invention shall have the meanings that are commonly understood by those of ordinary skill in the art. Further, unless otherwise required by context, nomenclatures used herein, and techniques of molecular biology and cell culture are those well-known and commonly used in the art.

Nevertheless, with respect to the use of different terms throughout the current specification, the following definitions more particularly apply.

By “transplantation”, “transplanting”, “graft” or “grafting”, it is meant herein the process of transferring a healthy organ or a portion thereof, tissue or cells to a subject in need thereof, i.e. to a subject of which the function of said organ, tissue or cells is deficient and may therefore compromise the long-term survival of said subject. A “renal transplantation” thus refers to the transplantation of a kidney, a portion thereof, kidney tissue or renal cells in a subject in need thereof. The term “transplantation” encompasses the introduction of any material suitable for such transfer, including, without limitation, natural materials, biological materials engineered in vitro, or synthetic materials. The term “transplant” thus refers to the material (organ, tissue or cells, which are natural, synthetic or engineered in vitro) to be transplanted. It should be further noted that said transplantation may be homologous (“homotransplantation” or “allotransplantation” or “allograft” or “allogeneic transplantation”), autologous (“autotransplantation” or “autograft”) or isogenic (isotransplantation), depending upon the origin of the material to be used. The term “allotransplantation” more specifically refers to a transplantation as defined above between individuals belonging to the same species. As for the term “autotransplantation”, it refers to a transplantation from one part of the body to another, within the same individual. For example, in the context of the present invention, a renal autotransplantation may consist in surgically transferring a kidney from the lumbar fossa to the iliac fossa, within the same individual. Finally, the term “isogenic transplantation” refers to a transplantation between identical twins. Preferably, the transplantation according to the invention is a homologous transplantation.

The term “subject” or “patient” is used herein to describe any member of the animal kingdom, preferably a human being. In a preferred embodiment of the invention, said patient is an end stage renal disease (ESRD) patient.

The term “prognosis”, “prognosing”, or “clinical outcome” as used herein refers to the likely outcome or course of a transplantation; such as the chance of long-term or short-term graft survival. A prognosis may indicate whether the grafted material is likely to be functional within the individual, and/or whether a patient in need of a transplantation is likely or not to reject the graft. The term “positive clinical outcome” or “good prognosis” means a desired clinical outcome. In the context of the present invention, a positive clinical outcome may be, for example, an expectation or low probability of having to dialyze the patient after transplantation, and/or an extended graft survival by comparison to a control population in which a renal transplantation would have an expected survival of preferably 5 years, more preferably of 10 years. Such positive clinical outcome is preferably linked to a long-term graft survival and/or functionality of the grafted material within the individual. By contrast, the terms “negative clinical outcome” and “poor prognosis” are used herein interchangeably to mean an undesired clinical outcome. In the context of the present invention, a negative clinical outcome may be, for example, an expectation or high probability of having to dialyze the patient after transplantation, and/or an expectation or high probability of graft rejection preferably within less than 10 years, more preferably within less than 5 years, after renal transplantation. Such negative clinical outcome is preferably linked to a short-term graft survival and/or low functionality of the grafted material within the individual.

The term “level” of a protein as used herein refers to an amount (e.g. relative amount or concentration) of said protein that is detectable or measurable in a biological sample of a subject. For example, the level can be a concentration such as μg/L or a relative amount by comparison to a reference level. The act of actually “determining the level” of a protein in a biological sample refers to the act of actively detecting whether the expression of said protein is upregulated, downregulated or substantially unchanged when compared to a reference level. Methods for measuring protein levels are well-known in the art and therefore need not be further detailed herein.

By “reference level” or “control level” of a protein, in particular of ferritin, it is meant a predetermined level of said protein, which can be used as a reference in the present invention. For example, a reference level can be the level of a protein, in particular of ferritin, in a biological sample of subject having a positive clinical outcome, or the average or median, preferably median, the protein level in a biological sample of a population of subjects having a positive clinical outcome.

The term “circulating ferritin level” thus means herein the level of the ferritin protein detectable or measurable within a body fluid of a subject. In the context of the present invention, a body fluid is preferably selected from blood, or a fractional component thereof such as serum, plasma, or a cellular extract thereof. Methods for determining circulating ferritin level are well-known in the art: they include, without limitation, Western blot, immunoblot, enzyme-linked immunosorbant assay (ELISA), enzyme-linked immunospot (ELISPOT), radioimmunoassay (RIA), immunohistochemistry or immunoprecipitation, and may be performed by the skilled practitioner thanks to any available commercial kit, such as the ferritin ELISA kit from Calbiotech (Spring Valley, Calif., USA), the ferritin immunoenzymatic kit from Biocheck, Inc (Foster City, Calif., USA), the ferritin ELISA kit from IBL Immuno-Biological Laboratories (Minneapolis, Minn., USA), or the Spectro Ferritin kit from Ramco Laboratories, Inc. (Stafford, Tex., USA).

As used herein, the term “ischemia/reperfusion injury” or “IRI” refers to cellular damage (notably necrotic cell death), that occurs to an organ or tissue due to restoration of blood flow following a period of ischemia. More particularly, the term “ischemia” refers to an inadequate blood supply (circulation) to a local area (i.e., organ or tissue) due to blockage of the blood vessels to said area. Ischemia includes complete interruption of blood flow and oxygen delivery to a tissue, as well as hypoxia whereby there is a substantial reduction in oxygen delivery to a tissue. The damage caused to an organ or tissue under ischemic conditions is then enhanced after restoration of the blood flow (i.e. “reperfusion”) with the formation of reactive oxygen species. In the context of the present invention, ischemia/reperfusion injury can be resulting from a renal transplantation, and can thus be referred herein as “renal transplant ischemia/reperfusion injury”. Such ischemia/reperfusion injury is known to be a contributing factor to delayed graft function and graft rejection (e.g. acute rejection, chronic rejection).

The term “preventing (or prevention) of renal transplant ischemia-reperfusion injury” means herein reducing the incidence of renal transplant ischemia-reperfusion injury. In other terms, prevention means that a patient has a decreased risk of developing a renal transplant ischemia-reperfusion injury following a renal transplantation.

On the other hand, the term “reducing (or reduction) of renal transplant ischemia-reperfusion injury” means herein reducing the cellular damages linked to a renal transplant ischemia-reperfusion injury in a patient, as described above, including inflammatory responses caused by post-ischemic injury.

Additional definitions are provided throughout the specification.

The present invention may be understood more readily by reference to the following detailed description, including preferred embodiments of the invention, and examples included herein.

The inventors have surprisingly discovered that an increased baseline level of circulating ferritin is associated with a favorable renal graft outcome. Monitoring the circulating ferritin level in patients receiving a renal transplantation can thus allow for a reliable and rapid indication of the clinical outcome, such as short-term or long-term graft survival, and/or good or poor graft function.

Accordingly, in a first aspect, the present invention relates to a method for prognosing a renal transplantation in a patient, comprising the steps of:
a) determining the circulating ferritin level in said patient prior to renal transplantation;
b) comparing the circulating ferritin level determined in step a) to a reference level; and
c) determining the renal transplantation outcome based upon the comparison of step b).

Preferably, the renal transplantation is a renal homologous transplantation, as defined above.

Such reference level may vary depending on the type of tested biological sample and/or the method used for determining the circulating ferritin level. However, for particular experimental conditions (same type of tested biological sample, same method for determining the circulating ferritin level, for the patient and a reference pool of patients), said reference level may be determined by the following features:
all or most of a reference pool of patients undergoing a negative clinical outcome of renal transplantation have a circulating ferritin level inferior to said reference level; and
all or most of a reference pool of patients undergoing a positive clinical outcome of renal transplantation have a circulating ferritin level superior to said reference level.

In that case, a circulating ferritin level determined in step a) significantly inferior to said reference level is indicative of a negative clinical outcome of renal transplantation. Alternatively, a circulating ferritin level determined in step a) significantly superior to said reference level is indicative of a positive clinical outcome of renal transplantation.

A circulating ferritin level significantly superior to a reference level corresponds to a level superior of at least more than 10%, particularly more than 15%, more than 30% and even more particularly more than 35% of said reference level.

A circulating ferritin level significantly inferior to a reference level corresponds to a level inferior of at least more than 10%, particularly more than 15%, more than 30% and even more particularly more than 35% of said reference level.

More particularly, if the tested biological sample is a serum sample, the reference level can be 600 ng/mL of ferritin.

According to a preferred embodiment of the above method, a circulating ferritin level determined in step a) significantly inferior to 600 ng/mL is indicative of negative clinical outcome.

Alternatively, in another preferred embodiment, a circulating ferritin level determined in step a) significantly superior to 600 ng/mL is indicative of a positive clinical outcome.

Indeed, the inventors have demonstrated, in a cohort of 147 kidney allograft recipients, that patients who received a renal transplantation can be stratified into two distinct populations based upon this specific reference level of circulating ferritin, and that this stratification was predictive of the outcome of kidney function 7 days, after transplantation. In particular, the inventors showed that patients for which the circulating ferritin level was above 600 ng/mL was correlated with increased eGFR_{MDRD}, eGFR_{CKD-EPI}, and ⁵¹Cr-EDTA clearance, while the patients for which the circulating ferritin level was below 600 ng/mL was correlated with decreased eGFR_{MDRD}, eGFR_{CKD-EPI}, and ⁵¹Cr-EDTA clearance. As well known to the skilled person in the art, the parameters eGFR_{MDRD} (estimated glomerular filtration rate using the modification of diet in renal disease equation), eGFR_{CKD-EPI} (estimated glomerular filtration rate/chronic kidney disease epidemiology collaboration), and ⁵¹Cr-EDTA clearance allow the measuring of glomerular filtration rate (GFR), which is a direct indication of kidney graft function) (Flamant et al., 2012).

As indicated above, the circulating ferritin level can be easily determined from a mere blood sample and any fraction thereof, such as plasma, serum or a cellular extract. According to a particularly preferred embodiment of the present invention, the circulating ferritin level is determined from a serum sample of the patient.

The inventors have further discovered that iron is a critical modulator of post-ischemic injury, and that an iron overload displays a protection effect against post-ischemic kidney injury by impairing macrophage inflammatory responses. Accordingly, iron overload may be used to prevent or improve renal transplant ischemia-reperfusion injury in graft recipients.

Thus, in another aspect, the present invention provides a method for preventing or reducing renal transplant ischemia-reperfusion injury (IRI) in a patient in need thereof, comprising the step of:

α) administering an effective amount of a biological material capable of increasing circulating ferritin level to said patient prior to transplantation.

Indeed, the inventors have demonstrated that HFE −/− mice, which present increased serum iron levels and transferrin saturation, were protected against IRI, and notably displayed a reduced inflammatory response to IRI (such as decreased levels of IL6, MCP-1 and TNF-α and decreased inflammatory cell infiltrates) compared to control mice. The inventors further showed that macrophages cultured in presence of supra-physiological iron concentration downregulated, among others, genes involved in inflammatory response and in hypoxia, and upregulated genes involved in anti-oxidant responses.

As used herein, the term “administering” means that a drug of interest is delivered or dispensed to a subject orally, transdermally, or parenterally such as by intravenous, subcutaneous, intramuscular, intrathecal or intraperitoneal injection. It is within the skill of the person in the art to select the mode of administration depending upon the nature of the biological material. Accordingly, in a preferred embodiment, said effective amount of biological material is administered orally, transdermally or parenterally. In a more preferred embodiment, said effective amount of biological material is administered intravenously.

In the context of the present invention, by “effective amount”, it is meant a prophylactic or therapeutic concentration of a drug of interest, i.e. a concentration of the biological material which is sufficient to prevent or reduce renal transplant ischemia-reperfusion injury as described above. One skilled in the art would readily understand from the data disclosed herein that said effective amount must be sufficient so that the circulating ferritin level in said patients is above 600 ng/mL. It is within the skill of the person in the art to determine such effective amount.

Thus, in a preferred embodiment, said patient has a circulating ferritin level significantly inferior to 600 ng/mL.

According to a preferred embodiment, the method further comprises prior to step a), the steps of prognosing a renal transplantation in said patient according to the method for prognosing a renal transplantation according to the invention. Particularly, said patient has a negative clinical outcome of renal transplantation.

By “biological material”, it is meant herein a chemical or organic material that can have a biological activity, and that may be used by a living organism for generation or maintenance of life and/or for the regulation the cellular metabolism. Such material includes, without limitation, cells or tissue, which can either be natural, synthetic or engineered in vitro, as well as components thereof such as nucleic acids, proteins, or chemical compounds. In the context of the invention, it is within the skill of the person in the art to identify a biological material which is capable of increasing the circulating ferritin level in a subject. Said material is preferably selected herein from the group consisting of iron, blood transfusion products, blood substitutes, antibodies directed against hepcidin, and any derivative thereof. Examples of blood transfusion products according to the invention include, without limitation, whole blood or purified red blood cells after leukoreduction, while blood substitutes include, without limitation, hemoglobin derivatives such as Hemopure, Oxyglobin or PolyHeme.

According to a preferred embodiment, said biological material is iron.

Should iron be administered to the patient in need, it shall be understood that said iron can be in any form, which is suitable for the purposes of the invention. For example, iron may be in the form of carbonyl iron; or of one or more ferrous salts, including iron gluconate, iron chloride, iron sulfate, iron fumarate, and ferrous aspartate, which are all equally effective. Iron may also be complexed to a sugar, such as iron sucrose (Venofer), iron polysaccharide (Niferex), iron dextran (Dexferrum, Imferon, INFeD), iron carboxymaltose (Ferinject), or iron oxide particles (Feraheme). It is well known in the art that iron complexed to a sugar is more suitable for parenteral administration, while ferrous salts and carbonyl iron are more suitable for oral administration. Still, alternatively, the iron may also be in the form of an iron-containing drug, which combines iron with another active compound such as a vitamin supplement, erythropoietin, or an erythropoietin stimulating agent. In a preferred embodiment of the present invention, the vitamin supplement is selected from the group consisting of thiamine, riboflavin, niacin, pantothenic acid, pyroxidine, biotin, folic acid, and cobalamin. One ordinary skilled person in the art would nevertheless understand that the therapeutic or prophylactic effective amount of iron to be used in the present invention is the one of elemental iron.

As exposed above, the inventors have discovered that iron is a critical modulator of post-ischemic injury, and that an iron overload displays a protection effect against post-ischemic kidney injury by impairing macrophage inflammatory responses. Accordingly, iron overload may be used in renal transplantation, particularly to prevent or improve renal transplant ischemia-reperfusion injury in graft recipients.

Thus, in another aspect, the present invention relates to a method for renal transplantation in a patient in need thereof, comprising the steps of:
α) administering an effective amount of a biological material capable of increasing circulating ferritin level to said patient prior to transplantation; and
β) performing a renal transplantation in said patient.

Said biological material to be administered is preferably as described above.

According to a preferred embodiment, said biological material is iron.

As indicated above, the term “administering” means that a drug of interest is delivered or dispensed to a subject orally, transdermally, or parenterally such as by intravenous, subcutaneous, intramuscular, intrathecal or intraperitoneal injection. It is within the skill of the person in the art to select the mode of administration depending upon the nature of the biological material. Accordingly, in a preferred embodiment, said effective amount of biological material is administered orally or parenterally. More preferable, said effective amount of biological material is administered intravenously.

Besides, as indicated above, in the context of the present invention, by “effective amount”, it is meant a prophylactic or therapeutic concentration of a drug of interest, i.e. a concentration of biological material which is sufficient to prevent or reduce renal transplant ischemia-reperfusion injury as described above. One skilled in the art would readily understand from the data disclosed herein that said effective amount of iron must be sufficient so that the circulating ferritin level in said patients is above 600 ng/mL. It is within the skill of the person in the art to determine such effective amount.

Thus, in a preferred embodiment, said patient has a circulating ferritin level significantly inferior to 600 ng/mL.

According to a preferred embodiment, the method further comprises prior to step a), the steps of prognosing a renal transplantation in said patient according to the method for prognosing a renal transplantation according to the invention as described above. Particularly, said patient has a negative clinical outcome.

Step β) of performing a renal transplantation in said patient can be carried out according to any methods well known by one skilled in the art (Perico et al., 2004) (Hariharan et al., 2000).

The present invention will be better understood in the light of the following detailed description of experiments, including examples. Nevertheless, the skilled artisan will appreciate that this detailed description is not limitative and that various modifications, substitutions, omissions, and changes may be made without departing from the scope of the invention.

### BRIEF DESCRIPTION OF THE DRAWINGS

FIG. 1: Iron overload protects against IRI in mice.

### HFE^{-/-} Mice are Protected Against IRI. (A-G)

129/Sv or HFE^{-/-} animals were subjected to bilateral ischemia or sham-operated and sacrificed 24 h following reperfusion. (A) serum creatinine and (B) blood urea nitrogen (BUN) levels in HFE^{-/-} and WT mice following IRI or sham surgery. n=5-19. Quantified results represent the mean±SEM from 5 independent experiments; *, P<0.05; **, P<0.01 and ***, P<0.001. (C) Representative Masson's trichrome staining of kidneys from WT and HFE^{-/-} mice after renal IRI or sham surgery. (D) Histological scores from C. Tissue injury was scored in cortical medullary junction areas (100× magnification). n=5-11 mice/group and ten fields of each kidney were evaluated. Values represent mean±SEM from 3 independent experiments; **, P<0.01. (E) Representative immunostaining for the detection of GR1⁺ or CD11b⁺ cell infiltrates in frozen sections of WT and HFE^{-/-} mice 24 h after IRI. (F) Quantification of GR1⁺ and CD11b⁺ cells in interstitial space (inter. space) from 10 randomly chosen fields (×400 magnification) for each mouse. n=4 mice per group. Values represent mean±SEM; *, P<0.05; ***, P<0.001. (G) Pro-inflammatory cytokine detection in kidney extracts from WT and HFE^{-/-} mice exposed to sham surgery or IRI. n=4 mice per group. Values are mean±SEM; *, P<0.05; **, P<0.01; ***, P<0.001.

Lack of HFE Expression on Bone Marrow-Derived Cells does not Prevent IRI (H and I).

(H) Irradiated WT mice were reconstituted with BM from WT (WT→WT) and HFE^{-/-} mice (HFE^{-/-}→WT). Eight weeks later, mice were subjected to IRI or were sham-operated. BUN and serum creatinine were assessed 24 h later; n=4-9 mice per group. Values are mean±SEM; *, P<0.05; **, P<0.01; ***, P<0.001. (I) Bone marrow cells from WT mice were used to reconstitute irradiated WT (WT→WT) or HFE^{-/-} (WT→HFE^{-/-}) mice. Eight weeks later, mice were subjected to IRI or were sham-operated. BUN and serum creatinine were assessed 24 h later; n=4-9 mice per group. Values are mean±SEM;*, P<0.05; **, P<0.01.

FIG. 2: Effect of iron load on macrophage function.

### Chronic Iron Injection Prevents Inflammatory Macrophage Recruitment (A-D).

Mice were intraperitoneally injected for 10 days with saline solution or iron sucrose solution (Venofer®, 90 μg/day) before LPS challenge. (A-C) Absolute numbers of small peritoneal macrophages (SPM) (A), large peritoneal macrophages (LPM) (B) and neutrophils (CD11b⁺Ly6G⁺) (C) in the peritoneal cavity of saline or Venofer® injected mice treated or not with LPS; n=4 mice for each group. Values are mean±SEM; *, P<0.05 **, P<0.01. (D) Representative dot plot analysis (gated on CD45⁺Ly6G⁻ cells) of inflammatory macrophages (SPM; CD11b^{low}F4/80^{low}Ly6G⁻Ly6C⁻) and resident macrophages (LPM; CD11b^{high}F4/80^{high}Ly6G⁻Ly6C⁺). Numbers adjacent to boxed areas indicate percent of SPM (lower gate) and LPM (upper gate).

### Impaired Inflammatory Responses in Macrophages Cultured in the Presence of Increased Iron Levels.

(E) Bone-marrow derived macrophages (BMM) were differentiated in the absence or in the presence of iron (FeCl3-NTA at 20 or 60 μM). At day 7 of culture, cells were stimulated overnight with LPS (150 ng/mL), poly-IC (15 μg/mL) or PGN (50 μg/mL), stained for CD11b and F4/80 macrophage markers and activation was evaluated by CD86 expression. Results are representative of 1 out of 4 independent experiments. Values are mean±SEM; *, P<0.05 **, P<0.01; and ***, P<0.001.

### Increased Iron Levels Leads to Impaired Inflammatory Responses in Macrophages.

(F) Production of TNF-α, MCP-1 and IL-6 measured in the culture supernatant of BMM or iron-BMM stimulated or not with LPS. n=4 experiments. Values are mean±SEM; **, P<0.01; and ***, P<0.001. Reactive oxygen species (ROS) production by macrophages cultured in the presence of different extracellular iron levels (G and H). Bone-marrow derived macrophages (BMM) were differentiated in the absence or in the presence of iron (FeCl3-NTA at 10 or 20 μM) for 7 days. At day 7 of culture, cells were stimulated or not for 3 hours with LPS (150 ng/mL), and ROS were measured by DCFH fluorescence and analyzed by flow cytometry. Because FeCl3-NTA was diluted in NaOH, the same concentration of NaOH was used as vehicle. (G) Flow cytometry data of cells differentiated with 20 μM FeCl3-NTA are presented as a histogram. (H) Flow cytometry data of independent cultured macrophages are presented. n=3 experiments. Values are mean±SEM; *, P<0.05.

FIG. 3: Effect of iron load on kidney injury.

Macrophages Treated with High Iron Levels are Unable to Reconstitute IRI in Clodronate Liposomes Treated Mice (A-C).

Mice were injected with 200 μl liposome containing PBS or clodronate 48 hours before surgery. Animals were then subjected to 40 min of bilateral ischemia or were sham-operated. At 1 hour of reperfusion, BMM cultured in the presence of 60 μM of iron (Fe-BMM) or control BMM were administrated by retro-orbital injection and mice were sacrificed at 24 h of reperfusion. Serum creatinine (A) and BUN (B) levels in control and reconstituted mice. n=6-8 mice. Values are mean±SEM; *, P<0.05; **, P<0.01; and ***, P<0.001. (C) Representative morphology (Masson's trichrome staining) of cortical-medullary junction from sham or IRI mice.

Increased Serum Ferritin Levels are Associated with the Outcome of Kidney Allografts (D-G).

Markers of iron homeostasis (serum iron (D), TSAT (E) and TIBC (F)), according to baseline serum ferritin levels. High ferritin group comprises patients within the 4th quartile (>600 ng/mL; n=38). Low ferritin group is composed of patients included in the 1^{st}, 2^{nd} and 3^{rd} quartiles (<600 ng/mL; n=109). (G) eGFR_{MDRD} at day 7 from RTx in low and high ferritin groups. eGFR_{MDRD}=Estimated glomerular filtration calculated with the modification of diet in renal disease formula (eGFR_{MDRD}=175×plasma creatinine^{-1.154}×age^{-0.203} (×0.742 if female) (×1.21 if black)) TIBC=Total iron binding capacity; TSAT=transferrin saturation. *, P<0.05; **, P<0.01; and ***, P<0.001.

### EXAMPLES

### 1. Material and Methods

1.1. Patients

Patients from Saint Louis and Bichat hospitals (APHP, Paris, France) who received kidney allografts from brain dead donors between Nov. 29, 2008 and Dec. 8, 2011 were considered for this study. Patients whose serum ferritin levels were assessed on the day of renal transplantation (RTx) were included. All patients gave their consent for scientific use of anonymous data. Patients were divided into two groups according to their baseline serum ferritin levels being in the fourth quartile (high ferritin group) or in the first, second and third quartiles (low ferritin group).

Recipients demographic (date of birth, date of transplantation, cause of ESRD, ethnicity, gender, dialysis modality) and biological data (serum concentrations of iron, ferritin, transferrin, transferrin saturation (TSAT), total iron binding capacity (TIBC), serum creatinine, cell blood count (CBC) and C-reactive protein (CRP)) were collected on the day of transplantation and serum creatinine levels on the seventh day after RTx. Donors (living or deceased-donor, donor age and gender, presence of reversible cardiac arrest, dual kidney transplantation) and transplantation characteristics (combined transplantations including kidney, delayed graft function and acute graft rejection before discharge) were collected from the clinical chart of eligible patients.

Estimated glomerular filtration rate (eGFR) on the seventh day after transplantation was calculated with the Modification of Diet in Renal disease study formula (eGFR_{MDRD})

Briefly, renal clearance of ⁵¹Cr-EDTA was determined after a single intravenous bolus injection of ⁵¹Cr-EDTA. After allowing 1 h for distribution of the tracer in the extracellular fluid, renal ⁵¹Cr-EDTA clearance was determined on 6 consecutive 30-min clearance periods. When timed urine samples could not be obtained or mean urine flow was below 1 ml/min, plasma clearance of ⁵¹Cr-EDTA was calculated using mono-exponential analysis with the Brochner-Mortensen correction. mGFR was normalized to body surface area which was calculated with the Mosteller formula.

1.2. Mice

All experiments were conducted with male mice. C57BL/6 (7-12 weeks of age, 20-25 g) were purchased from Charles River Laboratories. C57BL/6Ly5.1 congenic mice (B6.SJL-Ptprca Pep3b/BoyOrl) were purchased from TAAM-CNRS Orleans USP44. HFE^{-/-} and WT mice (12-14 weeks of age, 129/sv background) were bred and housed in the pathogen-free facilities of Bichat Medical School. All protocols were approved by the Animal Care Committee of INSERM.

1.3. Surgical Protocol

Animals were anesthetized by an intraperitoneal injection of ketamine (100 mg/kg)/and xylazine (10 mg/kg) solution. Animals (warmed with a warming blanket maintained at 37° C.) were subjected to median incision and both renal pedicles were cross-clamped for 40 (C57BL/6 background) or 45 minutes (129/sv background) The clamps were then removed; the flank incision was closed with 4-0 silk sutures. The animals received warm saline instilled into the peritoneal cavity during the procedure. Mice were returned to cages for 24 hours. After 24 hours of reperfusion, animals were re-anesthetized, blood was obtained by retro-orbital bleeding, and kidneys were removed for various analyses.

1.4. Generation of Chimeric Mice

The marrow from tibias and the femurs of donor mice (12 weeks of age, 23-25 g) was harvested. Bones were flushed under sterile conditions with RPMI 1640 (invitrogen) containing 10% FCS. Cells were washed, resuspended in PBS containing 1% BSA and viable cells were counted. Recipient WT and HFE^{-/-} animals (12 weeks of age) were lethally irradiated (9 Gy). Reconstitution was performed 4 hours after irradiation with 5·10⁶ BM cells. Chimeric mice were used 10 weeks after bone marrow transfer. The origin of donor or recipient cells was determined by congenic marker staining (Ly5.1 versus Ly5.2). In the conditions set up herein, reconstitution efficiency was nearly 98%. 3 groups of chimeric mice were generated:

WT_{129/sv}→WT_{Ly5.1}, WT_{Ly5.1}→WT_{129/sv}, HFE^{-/-}_{129/sv}→WT_{Ly5.1} and WT_{Ly5.1}→HFE_{129/sv}.

1.5. Peritonitis Model

Mice were intraperitoneally injected with 4.5 mg/kg Venofer® (iron-sucrose) or vehicule for 10 days. Peritonitis was induced by i.p injection of LPS (7.5 mg/kg) 2 hours after the last treatment. Animals were sacrificed 12 hours after LPS injection. 10 ml PBS containing with 0.5% BSA and 2 mM EDTA were then injected into peritoneal cavity. After injection, cells were dislodged by gentle massage of peritoneum. Peritoneal fluids were collected using a 21-g needle attached to a 10-ml syringe and washed.

1.6. In Vivo Depletion of Monocytes/Macrophages and Adoptive Transfer

Clodronate liposomes were purchased from Professor Nico van Rooijen of Vrije University, The Netherlands (www.clodronateliposomes.org). After warming and resuspension of the liposomes, a dose of 200 μl was administered intravenously by retro-orbital injection to induce monocyte/macrophage depletion. The control group was subjected to the same protocol, except liposomes containing PBS were injected. BMM (5·10⁶ cells per animal) were administered intravenously by retro-orbital injection at 1 hour of reperfusion.

1.7. Serum Iron and Kidney Functional Parameters

Iron, transferrin, ferritin, Urea and creatinine levels were measured in sera of mice using the AU400 chemistry analyzer (Olympus). Transferrin saturation was calculated by dividing serum iron concentration by total iron-binding capacity.

1.8. Histology and Immunohistochemistry

Paraffin-embedded kidney sections (4 μm in thickness) were stained by Masson trichrome. Morphological assessment was performed by an experienced renal pathologist (M.F.) who was blinded to experimental groups. Tubular necrosis score was established for each animal by assessing the intensity of tubular lesions (null, mild, important) in 10 randomly chosen fields in the corticomedullary junction. Global tubular injury scores were expressed in percentage. For immunohistochemistry, frozen kidney sections were incubated with biotinylated antibody against mouse CD11b (clone M1/70, BD biosciences) for 1 hour at room temperature, or antibody against mouse GR1 (clone RB6-8C5 BD biosciences) overnight at 4° C., following by incubation with biotinylated anti-rat IgG (SouthernBiotech) for 1 hour at room temperature. The antibodies incubation was followed by incubation with streptavidin-HRP for 30 min (Vectastain, ABC kit; Vector Laboratories, Burlingame, USA) which was revealed by immunoperoxidase reaction (Dako, Carpinteria, USA). Slides were mounted with the Eukitt mounting medium (Electron Microscopy Sciences) and observed under an upright microscope (DM2000; Leica) using the 1M50 software (Leica).

1.9. Preparation of Bone Marrow Derived Macrophages (BMM)

Bone Marrow derived macrophages (BMM) were prepared from the femur and tibias of C57BL/6 mice between 6 and 8 weeks of age. Bone marrow cells were cultured for 7-8 days at 37° C. and 5% CO2 in at 2×10⁶ cells/ml in RPMI 1640 medium (Invitrogen) containing 10% FBS (v/v; Biowest), penicillin/streptomycin and 15% supernatant of L929 cells (v/v) as a source of M-CSF in the presence or in the absence of FeCl3-nitrilotriacetate (FeCl3-NTA; 10-60 μM as indicated). In some conditions, lipopolysaccharide (LPS) (150 ng/ml) (Sigma-Aldrich), peptidoglycan (PGN) (15 μg/ml), Poly-IC (50 μg/ml) (all from cayla-Invivogen, Toulouse, France) were added 12 hours before BBMs analysis on day 8.

1.10. Leukocyte Counts

Blood samples were collected in EDTA-coated tubes and analyzed for leukocyte count on a MS9-5 Blood Analyzer (Melet Schloesing Laboratories) according to the manufacturer's instructions.

1.11. Flow Cytometry

Cells suspensions were prepared from cell culture or tissue and were incubated with anti-FcγR mAb 2.4G2 to block IgG receptors before cell staining for specific surface markers. Cells (1·10⁶) were then stained at 4° C. in PBS containing 1% BSA with antibodies against mouse CD11b (M1/70, BioLegend), F4/80 (BM8, eBioscience), CD86 (GL-1, BioLegend), Ly6G (1A8, BD Biosciences), Ly6C (HK1.4, BioLegend). The presence of macrophages (CD11b⁺F4/80⁺Ly6G⁻ cells) in the peritoneum was analyzed by flow cytometry.

1.12. Cytokine Production

Kidney tissues were homogenized in 300 μl of RIPA buffer supplemented with phosphatase and protease inhibitors cocktail (Sigma-Aldrich) for 30 s. Samples were cooled on ice for 30 min. Lysates were finally centrifuged at 10 000 g at 4° C. The cytokines IL-6, TNF-α, MCP-1, IL-10 and IL-12 were measured simultaneously by cytometric bead array (CBA) using mouse inflammation kit (according to the manufacturer's instruction (BD biosciences). Results were analyzed using FCAP array software. Proteins were quantified in supernatants by the bicinchoninic acid (BCA) method according to the manufacturer's instructions (Pierce). Cytokine concentrations were normalized to total protein concentration and expressed as picograms per milligram of total tissue protein.

Quantitative Real-Time RT-PCR

Total RNA was extracted from BMM (RNEasyPlus Mini kit, Qiagen) and quantified with a Nanodrop system. RNA quality was assessed by 260/280 nm ratio and agarose gel electrophoresis. Reverse transcription was carried out from 500 ng total RNA using the iScript reverse transcription Supermix (Bio-Rad, Hercules, Calif., USA). Real-time PCR was performed using SsoFast EvaGreen Supermix (Bio-rad). The following sequences of primers were obtained from Eurofins MWG Operon (Ebersberg, Germany):

IL-6 (forward)
(SEQ ID NO: 1)
5′-CCACGGCCTTCCCTACTTCA-3′,
IL-6 (reverse)
(SEQ ID NO: 2)
5-′GCCATTGCACAACTCTTTTCTCAT-3′;
TNF-α (forward)
(SEQ ID NO: 3)
5′-GTAGCCCACGTCGTAGCAAACCACC-3′,
TNF-α (reverse)
(SEQ ID NO: 4)
5-′TGGGGCAGGGGCTCTTGACG-3′;
βActin (forward)
(SEQ ID NO: 5)
5-′GGCTGTATTCCCCTCCATCG-3′,
βActin (reverse)
(SEQ ID NO: 6)
5-′CCAGTTGGTAACAATGCCATGT-3′;
GAPDH (sense)
(SEQ ID NO: 7)
5′-ACGGCAAATTCAACGGCACAGTCA-3′,
and
GAPDH (anti-sense)
(SEQ ID NO: 8)
5′-TGGGGGCATCGGCAGAAGG-3′.

Gene quantification was performed in duplicate using CFX96 PCR System (Bio-rad). Data were normalized to GAPDH and β-Actin values.

1.13. Reactive Oxygen Species (ROS) Assessment

Bone-marrow derived macrophages (BMM) were stimulated by LPS (150 ng/ml) to induce ROS production. After 3 hours, BMM were washed with PBS and 5 μM dichlorodihydrofluorescein-diacetate (DCFH-DA) was added and incubated for 20 min at 37° C. Cells were washed again and taken to flow cytometer to ROS analysis. FeCl3-NTA was used as mentioned before and NaOH was used as vehicle.

1.14. Statistical Analysis

For human studies Fisher's exact test or chi-square test were used as appropriate to analyze categorical data. Unpaired t-test or Mann-Whitney test were used as appropriate to analyze continuous numerical data. Pearson correlations were used to study interaction between eGFR_{MDRD} and ferritin levels in elderly patients. Multiple regression analysis of determinants of kidney allograft outcome were compared according to maximum likelihood criteria (an arbitrary cut-off value of 45 mL/min/1.73 m² was used to define either good or bad kidney graft function outcome). The model included ferritin levels, recipient age (years), gender donor, donor age (years) and cold ischemia time (minutes). All analyses were performed with SAS 9.1 software (SAS Institute, Cary, N.C.).

For experimental studies statistical analyses were performed with GraphPad Prism (version 5.0; GraphPad Software). Student's t-test was used for all comparisons. A p-value of <0.05 was considered significant.

### 2. Results and Discussion

2.1. Protection Against Post-Ischemic Injury and Inflammatory Responses in Mice Presenting Constitutive Iron Overload

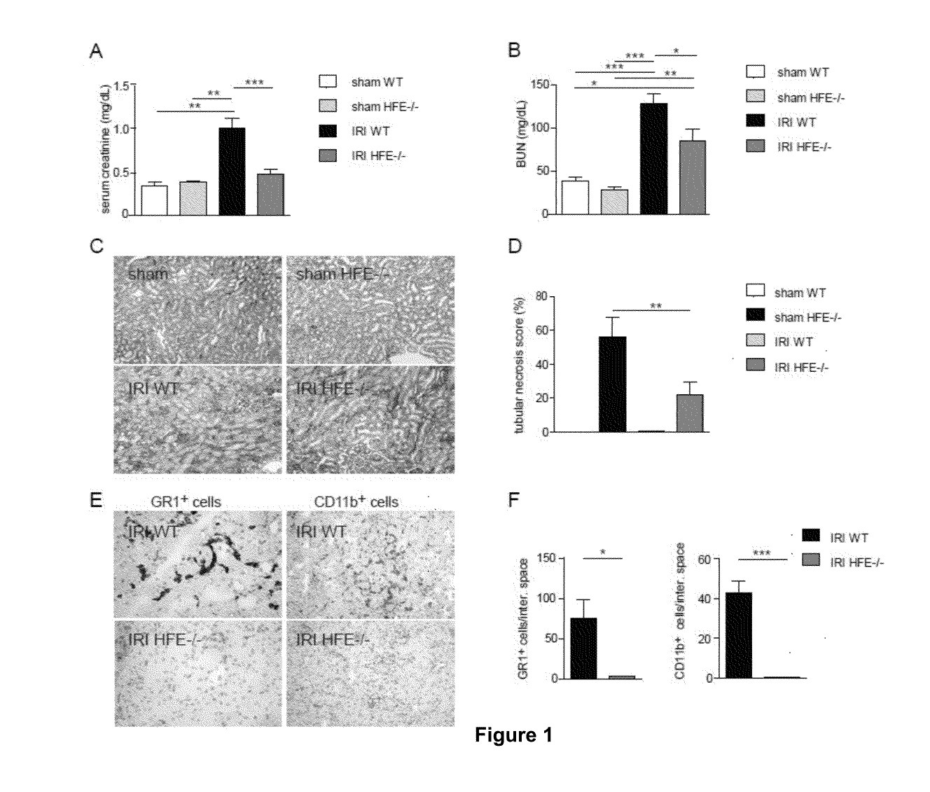

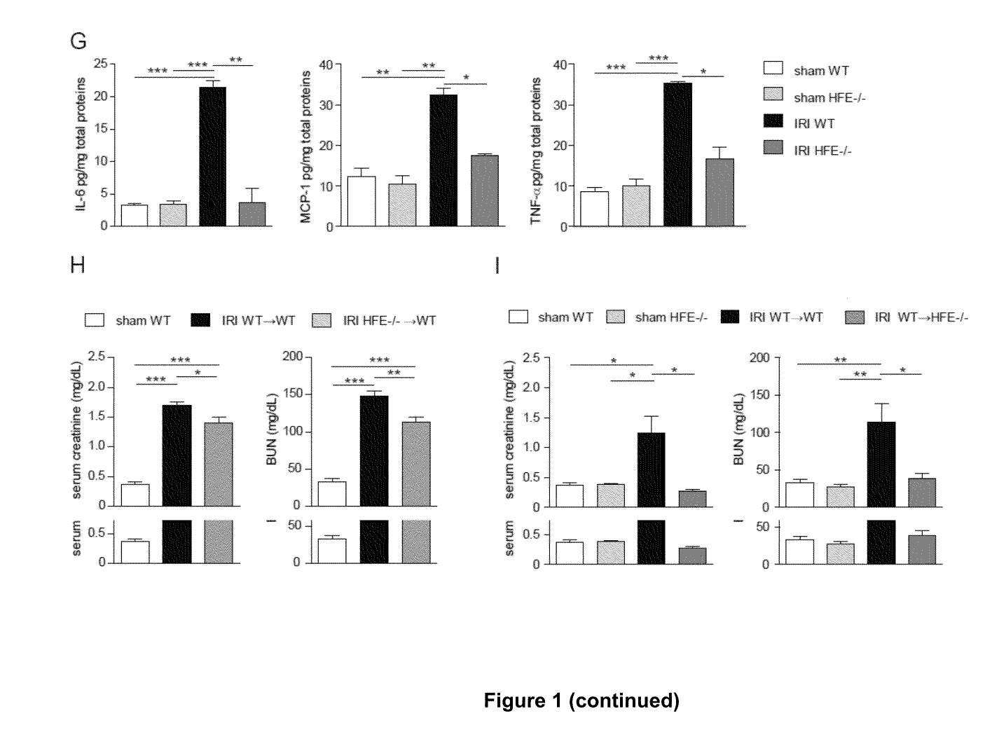

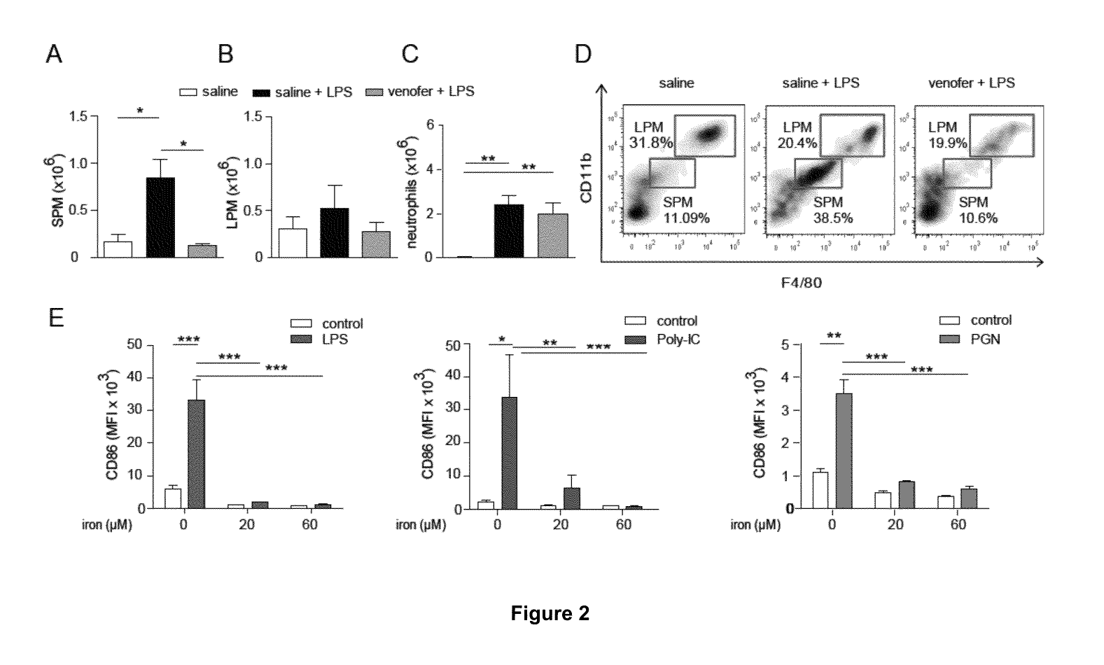

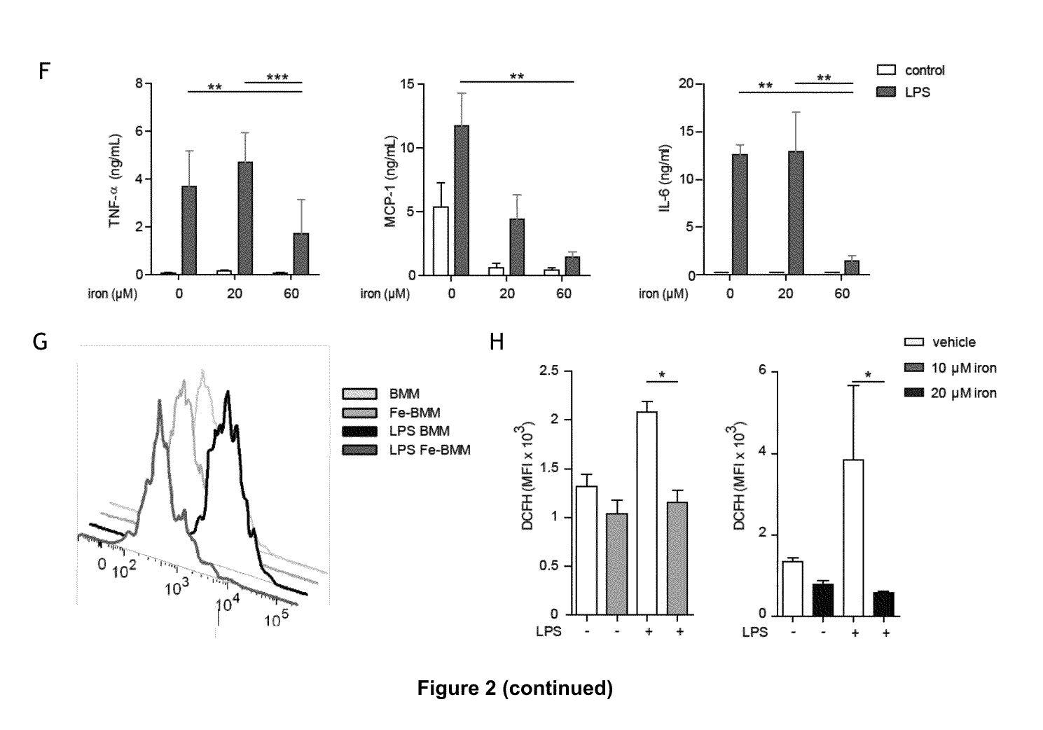

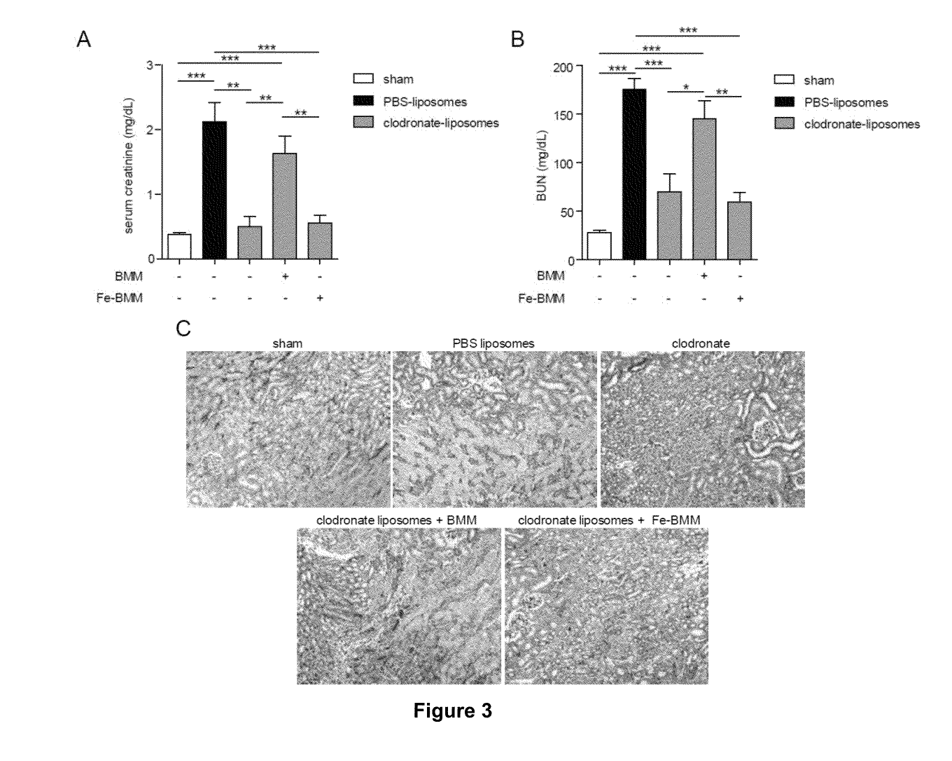

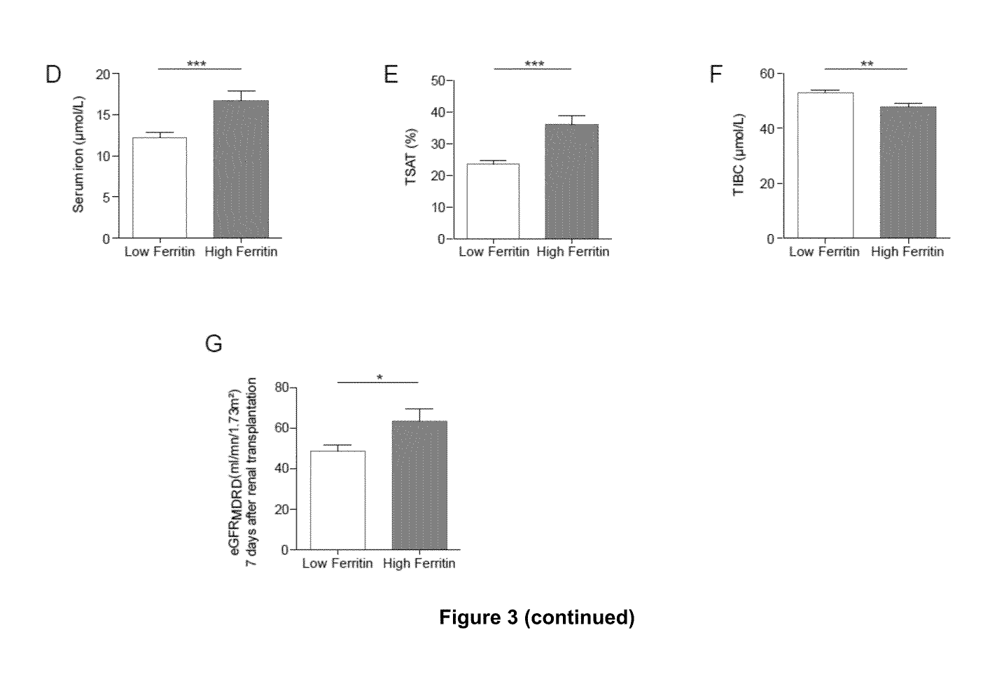

Intravenous iron is frequently used in CKD patients in order to improve responses to ESA (Locatelli et al., 2009). However, circulating iron levels can also interfere with inflammatory responses in monocytes (-Sonnweber et al., 201). The inventors aimed herein to study the impact of increased serum iron load on the outcome of ischemic kidney injury that reproduces conditions occurring during kidney transplantation (Bonventre et al., 2011; Duffield et al., 2005). HFE knockout mice (HFE^{-/-} mice) present progressive iron overload (Ajioka et al., 2002; Levy et al., 2000) and therefore constitute a valid model to address this question. The inventors subjected WT or HFE−/− mice to bilateral renal pedicle clamping (45 min) followed by 24 h reperfusion. Sham operated animals were used as controls. Analysis of renal parameters following post-ischemic reperfusion showed that WT (129/sv) mice presented increased serum creatinine and blood urea nitrogen (BUN) levels unlike HFE^{-/-} mice which were protected against the disease since both parameters were significantly reduced compared to wild-type controls (FIGS. 1A-B). This effect persisted after 48 h of reperfusion (data not shown). In agreement with kidney function data, histological evaluation and quantification of IRI lesions showed that whereas wild-type mice presented inflammatory cell infiltrates, tubular epithelial cell damage and dilated tubules with casts, HFE^{-/-} mice presented near to absent acute tubular necrosis (ATN) pathology (FIGS. 1C and D). Protection from AKI was restricted to ischemia-reperfusion since HFE^{-/-} mice were not protected against direct tubular damage induced by cisplatin (data not shown). Thus, modulation of iron homeostasis strongly impacts on the outcome of post-ischemic injury (data not shown).

Ischemic injury is a well-known model to study innate immune cell responses and sterile inflammation (i.e. inflammatory responses which are not triggered by microorganisms (Chen et al., 2010). Several studies using this model have shown that neutrophils and macrophages are key players promoting acute tissue injury (Bonventre et al., 2011; Rabb et al., 2006). As expected, recruitment of GR1⁺ and CD11b⁺ inflammatory cells was greatly increased in wild-type mice following acute ischemic injury (FIGS. 1E and F). In agreement, pro-inflammatory cytokine levels were increased after IRI compared with sham-operated control mice (FIG. 1G). In contrast to wild-type animals, HFE^{-/-} mice presented near to absent recruitment of inflammatory cells and decreased levels of IL-6, MCP-1 and TNF-α cytokine responses following IRI injury (FIGS. 1E-G). Finally, the mobilization of circulating inflammatory cells was greatly reduced in HFE^{-/-} mice compared to control mice suggesting that reduced responses in HFE^{-/-} mice derived from bone marrow myeloid cell mobilization (data not shown).

To determine whether the observed phenotype of HFE^{-/-} mice derived from the absence of HFE expression on monocyte/macrophages (Feder et al., 1996; Makui et al., 2005) or from iron overload (resulting from HFE deletion) the inventors performed bone marrow reconstitution experiments. Bone marrow reconstitution showed that HFE deficiency in myeloid cells was not sufficient to reproduce the phenotype of HFE^{-/-} animals since wild-type animals adoptively transferred with HFE^{-/-} bone marrow cells were only slightly protected against IRI (FIG. 1H). However, HFE-deficient animals reconstituted with HFE competent myeloid cells remained completely protected against IRI (FIG. 11). These observations further suggested that HFE deficiency in bone marrow-derived inflammatory cells does not mediate IRI protection in HFE-deficient animals. Altogether these results suggest that impaired inflammatory responses and protection against ischemic injury observed in HFE^{-/-} mice may involve iron overload.

2.2. Impact of Environmental Iron Load in Monocyte Inflammatory Responses

To selectively increase circulating iron levels in order to examine its impact on inflammatory responses mice were injected daily for 10 days with 90 micrograms of iron sucrose solution (Venofer®). The inventors next compared inflammatory responses between control mice (sterile saline-treated) and mice that had been repeatedly injected with iron (Venofer®-treated). For that, both groups of mice were injected with LPS (7.5 mg/kg) i.p. and the recruitment of myeloid inflammatory cells in the peritoneum was followed as previously described by Ghosn et al., 2010). Flow cytometry analysis of macrophage populations in the peritoneum showed that LPS induced an increase in inflammatory macrophages (small peritoneal macrophages, SPM, CD11b^{low}F4/80^{low}Ly6G⁻Ly6C⁻ cells) recruitment in PBS-treated mice (FIG. 2A). This recruitment was drastically decreased in Venofer®-treated mice (FIG. 2A). By contrast, iron treatment did not alter resident macrophage numbers (large peritoneal macrophages, LPM, CD11b^{high}F4/80^{high}Ly6 G⁻Ly6 C⁺ cells) or neutrophils (CD11b⁺GR1⁺ cells) recruitment following LPS injection compared to control mice (FIG. 2B-D). Therefore, in vivo chronic treatment with iron prevented inflammatory macrophage recruitment in a peritonitis model.

Erythrophagocytosis by Kupffer cells and spleen macrophages is a crucial mechanism involved in iron recycling in the organism (Andrews et al., 2008). Therefore, compared to other cell types, the machinery for dealing with iron homeostasis is optimized in these cells. To study the impact of environmental iron load on macrophage phenotype, bone marrow-derived macrophages (BMM) were differentiated in the absence or in the presence of soluble iron (Fe-NTA) at two concentrations (20 μM and 60 μM). Macrophage differentiation was not affected by culturing myeloid cells in the presence of different iron doses as assessed both in terms of cell morphology and of expression of cell surface markers (CD11b and F4/80) (data not shown).

To further investigate whether iron load could influence macrophage activation, the inventors analyzed responses to TLR agonists. Macrophages cultured in the presence of increased concentrations of iron were not able to up-regulate CD86 in response to TLR4 (LPS), TLR3 (poly I:C) and TLR2 (PGN) agonists (FIG. 2E). Increased iron levels in cultures promoted macrophage unresponsiveness to LPS both when it was added at the onset of cultures from primary bone marrow progenitors (day 0) or to cultures of almost completely differentiated cells (90% CD11b+ and F4/80+BMM; day 4) (data not shown). Thus, increased extracellular iron load impairs macrophage activation. To gain insight into the molecular mechanisms involved in iron load-dependent inhibition of macrophage inflammatory responses, the inventors performed comparative gene-chip analysis of macrophages treated or not with soluble iron (Fe-NTA, 60 μM) and subjected or not to LPS treatment. Venn diagram analysis from Affymetrix® microarray chips confirmed that the overall response to LPS differed between control (BMM) and iron-treated (Fe-BMM) macrophages (data not shown). Comparison Gene Ontology (GO) analysis was further performed to test the biological relevance of genes differing between these conditions. GO analysis showed that the 10 most significant pathways differing between BMM and Fe-BMM cells stimulated with LPS (LPS-BMM and LPS-Fe-BMM respectively) involved essentially genes implicated in immune function, healing and apoptosis (data not shown). As expected, genes implicated in inflammatory response were downregulated in LPS-Fe-BMM compared to LPS-BMM (data not shown). In agreement, genes involved in NF-κB responses were also down-regulated in LPS-Fe-BMM compared to LPS-BMM (data not shown). Accordingly, LPS-Fe-BMM showed impaired secretion of TNF-α, MCP-1 and IL-6 (FIG. 2F). Moreover, analysis of genes encoding markers of M1 and M2 (data not shown) showed that increased extracellular iron levels inhibited the expression of genes characterizing M1 or M2 phenotypes (Mosser et al., 2008).

Reactive oxygen species (ROS) are implicated in the control of inflammation (Groeger et al., 2010). Increased expression of genes involved in anti-oxidant responses were observed in Fe-BMM compared to control cells (data not shown) suggesting that molecular mechanism involved in the regulation of inflammatory responses by extracellular iron may involve ROS homeostasis. To evaluate whether overexpression of anti-oxidant genes resulted in impaired inflammatory responses the inventors measured ROS levels in control and Fe-BMM in response to LPS stimulation. ROS responses were impaired in LPS-Fe-BMM compared to control macrophages (FIGS. 2G and H). In conclusion, high extracellular iron levels impaired ROS responses to inflammatory stimuli and decreased pro-inflammatory cytokine secretion, activation markers expression and impaired NF-κB pathway activation in primary macrophages.

2.3. Macrophages Cultured with High Extracellular Iron Levels are Unable to Promote IRI

Macrophages are essential to IRI (Bonventre et al., 2011; Rabb et al., 2006) and macrophage depletion by clodronate liposomes abrogates IRI in mice (Day et al., 2005; Jo et al., 2006; Ferenbach et al., 2012). To further investigate whether macrophages cultured with increased load of extracellular iron were able to reproduce observations made in the HFE knockout model, the inventors reconstituted clodronate liposome-treated animals with macrophages derived in the presence of physiological and supra-physiological iron levels. As described previously by others, macrophage depletion abrogated post-ischemic injury in clodronate liposome-treated animals (FIG. 3A-B) (Day et al., 2005; Jo et al., 2006; Ferenbach et al., 2012). Whereas macrophages cultured in the presence of physiological iron levels reconstituted IRI, macrophages differentiated in the presence of increased iron levels did not reconstitute kidney injury (FIG. 3A-B). Histological analysis confirmed these findings (FIG. 3C). Therefore macrophages differentiated in the presence of increased iron concentrations were unable to reconstitute inflammation injury in macrophage-depleted animals subjected to ischemia-reperfusion.

2.4. Baseline Serum Ferritin Levels Correlate with Kidney Function in Allograft Recipients

To further validate these experimental observations, the inventors performed a retrospective cohort study and stratified a population of allograft recipients (n=147) according to their baseline ferritin levels, which constitute a reliable marker of iron load. The patients in the fourth quartile (serum ferritin levels >600 ng/ml, hereinafter called high ferritin group, n=38) were compared with patients who had normal or low serum ferritin levels (serum ferritin levels <600 ng/ml hereinafter called low ferritin group, n=109) (Table 1). Serum iron levels and serum transferrin saturation (TSAT) were increased and total iron-binding capacity (TIBC) was reduced in high ferritin group patients whereas C-reactive protein levels did not differ between the two groups confirming increased iron load in the high ferritin group (FIG. 3 D-F, and data not shown). Hemoglobin (Hb) levels were not different between the two groups (data not shown). Kidney function seven days post-transplantation was better in the high ferritin group than in the low ferritin group when estimated glomerular filtration rate (GFR) using the abbreviated Modification of Diet in Renal Disease equation (eGFRMDRD) (FIG. 3G) was considered. The inventors also noticed that patients in the high ferritin group were older which is usually associated with a worse graft outcome. Comparative analysis confirmed that other factors which could influence allograft function were not significantly different between high ferritin and low ferritin groups. These included: the age of recipient; the type of donor (living versus deceased donors); the cause of ESRD (diabetes, hypertension, GN, etc.) as well as other known risk factors for DGF (i.e. dual kidney transplantation, episodes of early acute rejection, donor age, donation after acute kidney failure and cold ischemia time) (Table 1).

TABLE 1
Baseline characteristics of kidney allograft according to ferritin levels before
transplantation. To convert to SI unit multiply ferritin by 1 to obtain μg/L.
Statistical analyses performed were Fisher's exact test for gender, donation after
circulatory death, dual kidney transplantation, donation after cardiac arrest, chi-
square test for donor type, dialysis mode, cause of ESRD, type of transplantation,
acute rejection episode, unpaired t-test for donor age and cold ischemia time, Mann-
Whitney test for recipient age at transplantation. Categorical data are expressed
as percent (numbers), donor age and cold ischemia time are expressed as mean ±
SE, recipient age is expressed as median (interquartile range).

| | | Low | High | |
|---|---|---|---|---|
| | Total | Ferritin | Ferritin | p |
| no | 147 | 109 | 38 | |
| Ferritin (ng/mL) | | 10 to 594 | 608 to 1798 | |
| Recipient characteristics | | | | |
| Gender (% Male (no)) | 57% (84) | 61% (66) | 47% (18) | 0.185 |
| Age (yr) | 49 ± 14 | 48 ± 14 | 53 ± 12 | 0.046 |
| Cause of ESRD. % (no) | | | | 0.750 |
| Diabetes mellitus | 24% (34) | 24% (25) | 24% (9) | |
| Glomerular disease | 17% (25) | 19% (20) | 13% (5) | |
| Hypertension | 16% (23) | 15% (16) | 18% (7) | |
| PKD | 12% (17) | 11% (12) | 13% (5) | |
| Congenital uropathy or reflux | 3% (4) | 4% (4) | 0% (0) | |
| Other known diagnoses | 14% (19) | 11% (12) | 18% (7) | |
| Unknown | 15% (22) | 15% (61) | 13% (5) | |
| Donor characteristic | | | | |
| Age (yr) | 51.2 ± 14.6 | 50.5 ± 14.7 | 53.3 ± 14.5 | 0.386 |
| Type of donation (% (no)) | | | | 0.600 |
| Living | 17% (23) | 16% (16) | 21% (7) | |
| Cadaveric | 83% (111) | 84% (84) | 74% (31) | |
| of which | | | | |
| risk of delayed graft function % (no) | 11% (12) | 12% (10) | 7% (2) | 0.727 |
| Donation after acute renal failure % (no) | 1% (1) | 0% (0) | 4% (1) | 0.193 |
| Dual kidney transplantation % (no) | 3% (3) | 4% (3) | 0% (0) | |
| Donation after cardiac arrest % (no) | 7% (8) | 8% (7) | 4% (1) | |
| Transplantation characteristic | | | | 0.516 |
| Transplanted organ (% (no)) | | | | |
| Kidney | 83% (122) | 83% (91) | 82% (31) | |
| Kidney-Pancreas | 10% (15) | 11% (12) | 8% (3) | |
| Other combined transplantation | 7% (11) | 6% (6) | 11% (4) | |
| Cold ischemia time (hours) | 15.2 ± 6.6 | 14.7 ± 15.6 | 16.7 ± 8.3 | 0.306 |
| Acute rejection % (no) | | | | 0.577 |
| Yes | 6% (9) | 6% (6) | 8% (3) | |
| No | 80% (117) | 82% (89) | 74% (28) | |
| | 14% (21) | 13% (14) | 18% (7) | |

Low Ferritin: Serum Ferritin <600 ng/mL; HIGH Ferritin: serum Ferritin >600 ng/mL.

According to multiple logistic regression analysis, ferritin levels predicted graft function 7 days (eGFRMDRD >45 ml/min/1.73 m²) (Table 2). In our retrospective cohort ferritin levels constituted a more reliable marker to predict allograft function than recipient age, sex gender of the donor, donor age and cold ischemia time, all known factors influencing graft outcome (Table 2) (Hariharan et al., 2000).

TABLE 2
Predictors of favorable kidney allograft outcome seven days
after transplantation using multiple logistic regression

| Variable | OR | 95% CI | P |
|---|---|---|---|
| Ferritin high | 5.702 | 1.714-18.973 | 0.005 |
| Recipient age | 0.946 | 0.885-1.012 | 0.107 |
| Male gender donor | 2.110 | 0.798-5.578 | 0.132 |
| Donor age | 1.029 | 0.969-1.094 | 0.349 |
| Cold ischemia time | 0.999 | 0.998-1.001 | 0.232 |

Favorable outcome defined as eGFR_{MDRD} >45 ml/mn/1.73 m²;
OR: odds ratio;
95% CI: 95% confidence interval

Levels of ferritin found in high-ferritin group exceeded all international standard recommendations for ESRD patients (Drueke et al., 2012; Eckardt et al., 2009; Locatelli et al., 2009; KDOQI Clinical practic guideline and clinical practice recommendation for anemia in chronic kidney disease, 2007). Altogether these results suggest that increased iron load is associated with improved short and intermediate-term graft function and that determination of ferritin serum levels could predict graft function.

### 3. Conclusion

In summary, the present study reveals a previously unsuspected role of serum iron levels in kidney ischemia-reperfusion injury. Increased iron levels are associated with more favorable outcome in kidney allograft recipients and in an experimental model of IRI. This protection is associated with the modulation of macrophage responsiveness to inflammatory stimuli. Under increased iron conditions, macrophages present impaired inflammatory responses to TLR triggering that paralleled their inability to reconstitute IRI lesions in macrophage-depleted mice. Hence, iron increased levels appear as a new negative regulator of macrophages responses in sterile inflammation of ischemic injury.

The findings reported herein have also clinical consequences in kidney transplantation. Indeed, baseline ferritin levels can be used as a new predictive marker of short and long-term renal allografts outcome.

### REFERENCES

Ajioka, R. S., Levy, J. E., Andrews, N. C., and Kushner, J. P. 2002. Blood 100:1465-1469.
Andrews, N. C. 2008. Blood 112:219-230.
Bhattacharyya, S., Ghosh, J., and Sil, P. C. 2012. Free Radic Res 46:1296-1307.
Bonventre, J. V., and Yang, L. 2011. J Clin Invest 121:4210-4221.
Day, Y. J., Huang, L., Ye, H., Linden, J., and Okusa, M. D. 2005. Am J Physiol Renal Physiol 288: F722-731.
Deb, S., Johnson, E. E., Robalinho-Teixeira, R. L., and Wessling-Resnick, M. 2009. Biometals 22:855-862.
Callens, C., Coulon, S., Naudin, J., Radford-Weiss, I., Boissel, N., Raffoux, E., Wang, P. H., Agarwal, S., Tamouza, H., Paubelle, E., et al. 2010. J Exp Med 207:731-750.
Chin, B. Y., Jiang, G., Wegiel, B., Wang, H. J., Macdonald, T., Zhang, X. C., Gallo, D., Cszimadia, E., Bach, F. H., Lee, P. J., et al. 2007. Proc Natl Acad Sci USA 104:5109-5114.
Drakesmith, H., and Prentice, A. M. 2012. Science 338:768-772.
Drueke, T. B., and Parfrey, P. S. 2012. Kidney Int 82:952-960.
Duffield, J. S., Park, K. M., Hsiao, L. L., Kelley, V. R., Scadden, D. T., Ichimura, T., and Bonventre, J. V. 2005. J Clin Invest 115:1743-1755.
Chen, G. Y., and Nunez, G. 2010. Nat Rev Immunol 10:826-837.
Eckardt, K. U., and Kasiske, B. L. 2009. Nat Rev Nephrol 5:650-657.
Feder, J. N., Gnirke, A., Thomas, W., Tsuchihashi, Z., Ruddy, D. A., Basava, A., Dormishian, F., Domingo, R., Jr., Ellis, M. C., Fullan, A., et al. (1996). Nat Genet; 13:399-408.
Ferenbach, D. A., Sheldrake, T. A., Dhaliwal, K., Kipari, T. M., Marson, L. P., Kluth, D. C., and Hughes, J. 2012. Kidney Int 82:928-933.
Flamant, M., Haymann, J. P., Vidal-Petiot, E., Letavernier, E., Clerici, C., Boffa, J. J., and Vrtovsnik, F. 2012. Am J Kidney Dis 60:847-849.
Ghosn, E. E., Cassado, A. A., Govoni, G. R., Fukuhara, T., Yang, Y., Monack, D. M., Bortoluci, K. R., Almeida, S. R., and Herzenberg, L. A. 2010. Proc Natl Acad Sci USA 107:2568-2573.
Groeger, A. L., Cipollina, C., Cole, M. P., Woodcock, S. R., Bonacci, G., Rudolph, T. K., Rudolph, V., Freeman, B. A., and Schopfer, F. J. 2010. Nat Chem Biol 6:433-441.
Hariharan, S., Johnson, C. P., Bresnahan, B. A., Taranto, S. E., McIntosh, M. J., and Stablein, D. 2000. N Engl J Med 342:605-612.
Herrero-Fresneda, I., Torras, J., Cruzado, J. M., Condom, E., Vidal, A., Riera, M., Lloberas, N., Alsina, J., and Grinyo, J. M. 2003. Am J Pathol 162:127-137.
Imtiyaz, H. Z., Williams, E. P., Hickey, M. M., Patel, S. A., Durham, A. C., Yuan, L. J., Hammond, R., Gimotty, P. A., Keith, B., and Simon, M. C. 2010. J Clin Invest 120:2699-2714.
Jo, S. K., Sung, S. A., Cho, W. Y., Go, K. J., and Kim, H. K. 2006. Nephrol Dial Transplant 21:1231-1239.
2007. KDOQI Clinical Practice Guideline and Clinical Practice Recommendations for anemia in chronic kidney disease: 2007 update of hemoglobin target. Am J Kidney Dis 50:471-530.
Levy, J. E., Montross, L. K., and Andrews, N. C. 2000. J Clin Invest 105:1209-1216.
Locatelli, F., Covic, A., Eckardt, K. U., Wiecek, A., and Vanholder, R. 2009. Nephrol Dial Transplant 24:348-354.
Makui, H., Soares, R. J., Jiang, W., Constante, M., and Santos, M. M. (2005). Blood; 106:2189-2195.
Mosser, D. M., and Edwards, J. P. 2008. Nat Rev Immunol 8:958-969.
Munoz, P., Zavala, G., Castillo, K., Aguirre, P., Hidalgo, C., and Nunez, M. T. 2006. Biol Res 39:189-190.
Nemeth, E., Rivera, S., Gabayan, V., Keller, C., Taudorf, S., Pedersen, B. K., and Ganz, T. 2004. J Clin Invest 113:1271-1276
Palevsky, P. M., Zhang, J. H., O'Connor, T. Z., Chertow, G. M., Crowley, S. T., Choudhury, D., Finkel, K., Kellum, J. A., Paganini, E., Schein, R. M., et al. 2008. N Engl J Med 359:7-20.
Perico, N., Cattaneo, D., Sayegh, M. H., and Remuzzi, G. 2004. Lancet 364:1814-1827.
Pham, C. G., Bubici, C., Zazzeroni, F., Papa, S., Jones, J., Alvarez, K., Jayawardena, S., De Smaele, E., Cong, R., Beaumont, C., et al. 2004. Cell 119:529-542.
Rabb, H. 2006. J Am Soc Nephrol 17:604-606.
Sindrilaru, A., Peters, T., Wieschalka, S., Baican, C., Baican, A., Peter, H., Hainzl, A., Schatz, S., Qi, Y., Schlecht, A., et al. 2011. J Clin Invest 121:985-997.
Tilney, N. L., and Guttmann, R. D. 1997. Transplantation 64:945-947.
Valderrabano, F., Jofre, R., and Lopez-Gomez, J. M. 2001. Am J Kidney Dis 38:443-464.
Yuan, X., Cong, Y., Hao, J., Shan, Y., Zhao, Z., Wang, S., and Chen, J. 2004. J Mol Biol 339:131-144.

1. A method for prognosing a renal transplantation in a patient, comprising the steps of:
a) determining the circulating ferritin level in said patient prior to renal transplantation;
b) comparing the circulating ferritin level determined in step a) to a reference level; and
c) determining the renal transplantation outcome based upon the comparison of step b).

2. The method according to claim 1, wherein a circulating ferritin level determined in step a) significantly inferior to 600 ng/mL is indicative of a negative clinical outcome.

3. The method according to claim 1, wherein a circulating ferritin level determined in step a) significantly superior to 600 ng/mL is indicative of a positive clinical outcome.

4. The method according to claim 1, wherein the circulating ferritin level is determined from a serum sample of said patient.

5. A method for preventing renal transplant ischemia-reperfusion injury in a patient in need thereof, comprising the step of:
α) administering an effective amount of a biological material capable of increasing circulating ferritin level to said patient prior to transplantation.

6. The method according to claim 5, wherein said biological material is preferably selected from the group consisting of iron, blood transfusion products, blood substitutes, antibodies directed against hepcidin, and any derivative thereof.

7. The method according to claim 5, wherein said patient has a circulating ferritin level significantly inferior to 600 ng/mL.

8. The method according to claim 6, wherein said iron is administered orally, transdermally or parenterally.

9. The method according to claim 6, wherein said iron is administered intravenously.

10. The method according to claim 5, further comprising, prior to step a), the prognosis of a renal transplantation in said patient according to the method of claim 1.

11. A method for renal transplantation in a patient in need thereof, comprising the steps of:
α) administering an effective amount of a biological material capable of increasing circulating ferritin level to said patient prior to transplantation; and
β) performing a renal transplantation in said patient.

12. The method according to claim 11, wherein said biological material is preferably selected from the group consisting of iron, blood transfusion products, blood substitutes, antibodies directed against hepcidin, and any derivative thereof.

13. The method according to claim 11 wherein said patient has a circulating ferritin level significantly inferior to 600 ng/mL.

14. The method according to claim 12, wherein said iron is administered orally, transdermally or parenterally.

15. The method according to claim 12, wherein said iron is administered intravenously.

16. The method according to claim 11, further comprising, prior to step a), the prognosis of a renal transplantation in said patient according to the method of claim 1.


SEQUENCE LISTING (8 sequences)

SEQ ID NO: 1 - DNA, 20 bp, Artificial Sequence
  IL6 forward primer
ccacggccttccctacttca

SEQ ID NO: 2 - DNA, 24 bp, Artificial Sequence
  IL6 reverse primer
gccattgcacaactcttttctcat

SEQ ID NO: 3 - DNA, 25 bp, Artificial Sequence
  TNF-alpha forward primer
gtagcccacgtcgtagcaaaccacc

SEQ ID NO: 4 - DNA, 20 bp, Artificial Sequence
  TNF-alpha reverse primer
tggggcaggggctcttgacg

SEQ ID NO: 5 - DNA, 20 bp, Artificial Sequence
  Beta-Actin forward primer
ggctgtattcccctccatcg

SEQ ID NO: 6 - DNA, 22 bp, Artificial Sequence
  Beta-Actin reverse primer
ccagttggtaacaatgccatgt

SEQ ID NO: 7 - DNA, 24 bp, Artificial Sequence
  GAPDH sense primer
acggcaaattcaacggcacagtca

SEQ ID NO: 8 - DNA, 19 bp, Artificial Sequence
  GAPDH anti-sense primer
tgggggcatcggcagaagg